SEQUENCE LISTING (2 sequences)

SEQ ID NO: 1 - protein, 15 aa, Carica candamarcensis
YPGSVDWRZKGAVTP

SEQ ID NO: 2 - protein, 43 aa, Carica candamarcensis
YPGSVDWRQKGAVTPVKDQNPCGSCWAFSTVATVEGINKIVTG

(19) United States
(12) Patent Application Publication
(10) Pub. No.: US 2003/0223984 A1
(43) Pub. Date: Dec. 4, 2003

(54) Proteases from carica having mitogenic activity and their methods of use

(76) Inventors: Carlos E. Salas, Bairro Bandeirantes (BR); MARIAM T. P. Lopes, Bairro Bandeirantes (BR); Abraham V. Schnidermann, Coquimbo (CL)

Correspondence Address: David G. Rosenbaum, ROSENBAUM & ASSOCIATES, P.C., Suite #3600, 875 North Michigan Avenue, Chicago, IL, 60611, US

(21) Appl. No.: 10/162,267

(22) Filed: Jun. 3, 2002

(51) Int. Cl. A61K 38/48; C12N 9/50

(52) U.S. Cl. 424/94.64; 435/219

(57) ABSTRACT

Proteases having mitogenic activity isolated from the genus Carica are provided. In particular the proteases are cysteine proteases isolated from Carica candamarcensis. In addition, the recombinant forms of the protease, including fragments and mutants with substantial homology are provided. Also provided are pharmaceutical compositions useful for treating wounds that include the disclosed proteases with mitogenic activity. A method of treating wounds is provided using the disclosed proteases.

### FIELD OF INVENTION

The present invention relates to a group of proteolytic enzymes or proteases isolated from the genus Carica. In particular, the proteolytic enzymes are cysteine proteases that function as mitogenic stimulators of mammalian cells. The present invention also relates to a process for the production of these enzymes and their use as a wound healing promoter.

### BACKGROUND

The skin is an important organ for homeostasis and host defense against foreign invaders. Specifically, it acts as the body's first line of defense against infection. Accordingly, it is important that lesions or wounds in the skin be rapidly closed to prevent infection. Some types of wounds, however, are resistant to healing under normal physiological conditions.

The process of wound healing involves a complex system of local and remote (systemic) resources. For example, amino acids and sugars are needed as substrates for collagen and proteoglycan synthesis. Migration of fibroblasts and epithelial/endothelial cells during the wound healing process places additional systemic demands on a subject during the wound healing process. Wounded tissues have unique nutritional needs and physiological features. Lymphocyte participation in wound healing has been demonstrated. Alteration in the hosts T-cell dependent immune response has also been shown to influence wound healing. Cyclosporine and anti T-cell antibodies, both of which interfere with T-cell function, abrogate wound healing. Similarly, macrophages and their products are also involved in wound healing. Increased circulation usually results in rapid delivery of monocytes and PMN's to the wound site. This in turn results in the elimination of bacterial contamination of the wound due to nonspecific killing mechanisms and also enhances the rate of wound healing. These various cell types are synthesized by the bone marrow.

While wound healing is typically an efficient and natural process that normally requires no special treatment, chronic non-healing wounds can occur. In the chronic cases, there is some underlying factor preventing healing and intervention is often necessary to complete the healing process. For example, pressure sores are initially acute wounds caused by ischaemic death of tissue due to excessive pressure and will usually heal readily when pressure is relieved and the blood supply restored. Often times it is difficult to resolve these causative factors and chronic wounds can develop. Most of these chronic wounds are characterized by the accumulation of devitalized tissue and cellular exudates at the outer surface. These products result from a restriction of nutrients to the damaged epithelium and form either a dry, hard eschar or, as in the case of deep moist wounds, a slough that frequently hardens on the outside with exposure to the air. The accumulation of these products in the wound bed is generally regarded to prevent or delay granulation and epithelialisation. The removal of this tissue by a process termed debridement is therefore thought to facilitate healing.

Debridement can be accomplished by both mechanical and non-mechanical methods. The mechanical methods require the physical elimination of the devitalized tissue from the healthy, but this difficult and often results in the aggravation of the wound. There are various non-mechanical debridement methods that include enzymes, hydrogels and chemical formulations. While various methods of debridement exist, there is no proven reliability of any particular method of debridement with respect to a particular wound. In particular, use of proteolytic enzymes in the early debridment (digestion and separation) of eschar tissues, such as in burn wounds, decubitus ulcers, pressure necroses and bed sores has been researched, e.g., streptokinase, trypsin and papain.

There remains a need for isolating and providing an agent that acts as an effective promoter of wound healing.

### SUMMARY OF INVENTION

Aspects of the present invention satisfy the unmet needs in the art, as disclosed above. In particular, the invention provides a natural protease isolated from Carica, or its metabolites, along with recombinant forms of the natural protease, including fragments and mutants, that exhibit protease activity comparable to the wild type protease.

Another aspect of the present invention provides a pharmaceutical composition comprising an amount of protease effect for wound treating, wherein the protease comprises one of the proteins disclosed herein, which includes the natural, recombinant, fragment, and mutant forms of the protease.

In another aspect of the present invention, a method of treating wounds is provided using the compositions disclosed herein. The compositions containing the proteins of the present invention are used for treating various wounds, including chronic wounds like ulcers.

Still another aspect of the present invention provides a protease having mitogenic or proliferative activity. The protease is preferably a cysteine protease and more preferably a protease isolated from Carica, particularly Carica candarmacensis.

These and other aspects of the invention will be obvious to those of ordinary skill in the art considering the provided disclosure and examples.

### DETAILED DESCRIPTION OF PREFERRED EMBODIMENTS

The present invention relates to proteases, more particularly, cysteine proteases, and the production and use thereof. In particular, the one aspect of the present invention relates to a group of cysteine proteases, termed CC23a-e. This group of cysteine proteases is characterized as immunologically distinct from papain, having a molecular mass of about 23,000 Da and having a distinct net charge.

In another aspect of the present invention, a process for the production of the disclosed cysteine proteases is provided comprising separation of the protease from latex or leaf plant material of Caricaceae and purification therefrom, including Carica candarmacensis and Carica papaya.

The disclosed cysteine proteases can be used to enhance the process of debridement and wound healing of eschar tissue resulting from burn wounds, sores and ulcers, for example. Another aspect of the present invention comprises an enzyme preparation specifically adapted for use in enhancing the process of debridement and wound healing. Another aspect of the present invention provides a pharmaceutical composition which comprises at least one of the present cysteine proteases CC23a-e and at least one pharmaceutically-acceptable carrier.

As indicated above, the cysteine proteases CC23a-e have a relative molecular mass of 23,000 Da and are highly basic proteins, having a pI greater than 9.5, and exhibit faster migration in a cathodal electrophoresis system. CC23a-e require thiol compounds for full activity and is inhibited by the class-specific inhibitors E-64 [L-3-carboxy-2,3-trans-epoxypropionyl-leucylamido (4-guanidino) butane] and chicken cystatin.

These cysteine proteases may be obtained by conventional preparative fast protein liquid chromatography (FPLC) cation-exchange chromatography at pH 9.2. Currently, commercially available crude latex extract powders (Technologic Farm) are commonly used as a source of the present proteases. More specifically, an extract may be chromatographed using a cation exchanger (Mono S) using a Pharmacia FPLC system and a gradient of 0.005M to 1.0M sodium chloride at pH 9.0. A variety of substrates may be used to assay the fractions for cysteine protease activity. The different proteases can be identified on the basis of their different substrate specificities.

The proteases can also be produced using recombinant DNA techniques. The sequence of a cysteine protease, e.g., CC23a, is determined using known protein sequencing techniques, including Edman degradation. Based on the determined amino acid sequence, DNA primers are synthesized. These DNA primers can be used in RT-PCR reactions to select the cDNA which code for this particular cysteine protease. Once the identity is confirmed, the recombinant cDNA can be cloned into an appropriate expression vector using well-known cloning techniques. The resulting expression vector can be used to transform an appropriate cell line to express the recombinant cysteine protease, which can be isolated using standard purification techniques. The proliferative property of the recombinant cysteine protease can be tested as done with the naturally derived protease.

Preferred mutants or fragments of the cysteine proteases disclosed herein have corresponding amino acid sequences, in relation to the natural amino acid sequence, that are substantially homologous. Substantially homology is used to describe amino acid sequences that have near identity to the naturally occurring protease but contain substitutions that do not greatly alter function. Specifically, substantially homologous means a protein having a sequence that has at least about 80%, usually at least about 90% and more usually at least about 98% sequence identity with the sequence of the disclosed cysteine protease, as measured by BLAST.

Site-directed mutagenesis can be used to create mutations of the proteases that still retain proliferative activity. Preferably, conservative mutations are contemplated. The conservative substitutions can be introduced by modification of DNA encoding for the polypeptides of the invention. A conservative substitution is recognized in the art as a substitution of one amino acid for another amino acid that has similar properties, typically allowing the expression of a functional protein.

The present cysteine proteases may be admixed with pharmaceutically-acceptable carriers for application to patients. The generally preferred route of administration is by topical application to the site of a wound or ulcer. The pharmaceutical preparation may be in the form of a sterile solution that is applied to an inert dressing, such as a gauze pad, a gel, or ointment that is placed directly on the wound.

Such pharmaceutical compositions may contain the cysteine protease(s) in an active form, or, preferably, in an inactive form in which the active site cysteine residue forms half of a disulphide bridge. The other half comprises a pharmaceutically-acceptable thiol compound, for example the amino acid cysteine. The present formulations may be prepared immediately before use by mixing a lyophilised preparation of the cysteine protease with an aqueous solution. If an enzyme is present in an inactive form, an activating agent, for example cysteine, must be added to regenerate the free active site thiol of the protease.

The cysteine proteases described herein are proliferative factors useful for enhancing the healing of wounds. An amount of the protease effective for wound healing is readily determined by one of ordinary skill in the art using standard techniques, and such an amount is applied to the wound by standard techniques known in the art. Preferably, the amount of protease effective for wound healing is a concentration ranging from about 50 ng/ml to about 500 ng/ml as a single application, or in dosing regimens that range from several times per day to once every few days for a period of one to several weeks. In a topical formulation, the amount effective for wound healing is about 0.01 μg/cm² to about 100 μg/cm² of cysteine protease administered directly to the wound. These cysteine proteases can be used to treat many types of chronic non-healing wounds, such as fall-thickness dermal ulcers, e.g., pressure sores, venous ulcers, and diabetic ulcers; to treat acute wounds such as burns, incisions, and injuries; and to speed the healing of wounds associated with reconstructive procedures such as skin grafting and flap placement, e.g., in the repairing of wounds and aiding cosmetic procedures. In addition, the cysteine proteases can be used to treat damage to the gastric epithelium, the lung epithelium, and other internal epithelial layers.

In cases where the cysteine proteases of this invention are being used for surface wound healing, they can be administered by topical means. For topical administration, the cysteine proteases are applied directly to the site of injury as a solution, spray, gel, cream, ointment or as a dry powder. Slow release devices directing these cysteine proteases to the injured site can be used. In addition, the cysteine proteases can be combined with topical bandages, or dressings, or sutures/staples, and with topical creams and ointments. In specific, the cysteine proteases of this invention can be used at a concentration ranging from about 50 ng/ml to about 500 ng/ml as a single application, or in dosing regimens that range from several times per day to once every few days for a period of one to several weeks. Usually, the amount of topical formulation administered is an amount which applies about 0.01 μg/cm² to about 100 μg/cm² of cysteine protease to the wound.

Cysteine proteases of this invention can also be used for in vitro culturing of responsive cell types, e.g., fibroblasts or epithelial cells. For such uses, the cysteine proteases can be added to the cell culture medium at a concentration of about 10 ng/ml to about 100 ng/ml. In addition, cells grown under growth factor stimulation can be used as a source of expanded cell populations for grafting purposes. For all of these applications, the cysteine proteases of this invention may be used alone or in combination with other proliferative factors, debriding agents and biologically active agents. Other debriding agents include trypsin, collagenase dextranomer, cadexomer iodine, and hydrogels, e.g., INTRASITE GEL® (Smith & Nephew Healthcare Ltd), STERIGEL® (Seton Healthcare Group plc) and GRANUGEL® (CovaTec UK, Ltd.).

### EXAMPLE 1

### Purification of CC23a-e from Latex Extract of Carica candarmacensis

Crude latex extract from Carica candarmacensis was dissolved in 1 M sodium acetate buffer, 1 mM EDTA, 0.01% sodium azide, pH 5.0. This was filtered through 0.22 μm filters and the protein content was determined by absorption at 280 nm using an A_{1%,280} of 20.1 (Murachi, T. and Yasui, M., Biochemistry, 4, 2275-2282, 1965). The preparation was then chromatographed on a G-10 filtration column (Amersham-Pharmacia) and eluted with the same buffer. The first peak corresponded to the bulk protease activity and was pooled and applied onto a CM-Sephadex column (Amersham-Pharmacia). The protein fractions were eluted with a linear gradient of 0.05M to 1.0M sodium acetate, pH 5.0 (essentially as described previously for chymopapain in Buttle, D. J., and Barrett, A. J., Biochem. J., 223, 81-88, (1984)). The second protein peak containing the protease activity termed CC23a-e was pooled and applied onto a Mono S HR 10/10 column of a FPLC system (Pharmacia). The protein mix was eluted with a gradient of 0.002M to 1.0M NaCl at pH 9.2.

Plots of A₂₈₀ and gradient composition were provided automatically. The fractions were stored, tightly capped at 4° C. until assayed and further processed. The five peaks eluting between 0.02M and 0.39M NaCl were taken and designated as peaks CC23a-e. Each of the peaks CC23a-e were concentrated and dialyzed in an Amicon PM10 concentration chamber against 10 mM sodium acetate, 1 mM EDTA, 0.01% sodium azide, pH 6.5 at 4° C.

### EXAMPLE 2

### Proliferation Assays

The above purified proteins, CC23a-e, their metabolites, or the crude extracts from which were derived, were assayed for their proliferative activity as follows. The mitogenic fractions were assayed as described below, using either fibroblasts (i.e., L929), epithelial cells (i.e., human keratinocytes), or human mammary cells (i.e., MDA MB 231). Fractions (5-50 ng/ml) were tested for proliferative activity by measuring the effect of aliquots of the fractions on DNA synthesis. This was accomplished by actually measuring the proliferation of L929, MDA MB 231 and keratinocytes cells by monitoring the incorporation of [³H]-thymidine into DNA and/or by measuring the increase in cell number, using MTT as an indicator.

Cells were plated at 1-2×10⁴ cells/well (Costar, Cambridge, Mass.) in RPMI 1640 (GIBCO, Grand Island, N.Y.) supplemented with 10% fetal calf serum (GIBCO, Grand Island, N.Y.) and antibiotics (100 units/ml penicillin and 100 μg/ml streptomycin sulfate, GIBCO, Grand Island, N.Y.). After 24-48 h at 37° C., 2,5% CO₂, cells were washed twice with phosphate-buffered saline, and the medium was replaced with 0.5% FBS RPMI 1640 supplemented with antibiotics for 24-36 h before the assay.

Test samples were then added at the specified concentrations during 24 to 72 h. One μCi [³H]-thymidine/well (Amersham Pharmacia) was added and remained in the assay for 18 h before arresting the reaction. The reaction was stopped by 2 washes with RPMI, 2 washes with 5% TCA and 2 washes with 95% ethanol. The remaining radioactivity was measured following treatment with Aquasol (Amersham-Phamacia) in a scintillation counter. The following table, Table 1, summarizes the proliferative effect of CC23a-e fractions on L929 cells.

TABLE 1

| Protein tested | 10 ng/mL | 50 ng/mL |
|---|---|---|
| Crude Fraction | 105% | 31% |
| CC23a | 69% | 98% |
| CC23b | 54% | 67% |
| CC23c | 18% | 16% |
| CC23d | 8% | 18% |
| CC23e | 61% | 32% |

The determined proliferative activity (assayed as [³H]-thymidine incorporation into L929 cells) showed that the purified form of CC23a-e was not destroyed by heating to 90° C. for 5 minutes but was significantly destroyed (60%) by exposure to the cysteine protease inhibitor E64 for a period of 30 min (data not shown).

As shown in Table 1, the purified protein CC23a stimulated close to 100% proliferation of L929 fibroblast cells at a concentration of 50 ng/ml, while the maximal stimulatory effect of CC23e was 18% at 50 ng/ml. Papain from Sigma, but not chymopapain, showed 25% stimulatory affect at 20 ng/ml using the same protocol. The proliferative effect of each protease tested was also reduced by the addition of the inhibitor E64.

### EXAMPLE 3

### N-Terminal Sequencing

The amino acid sequence of the purified form of the protease CC23a was determined. Approximately 1.7 μg of protein, obtained after cation exchange-MonoS chromatography, was loaded onto an Applied Biosystems gas-phase protein sequencer. Fifteen or forty-two rounds of Edman degradation were carried out, and identification of amino acid derivatives were made with an automated on-line PTH-amino acid analyzer (model 477A, Applied Biosystems, Foster City, Calif.).

The fifteen rounds of Edman degradation of CC23b and CC23c resulted in the identification of the first 15 N-terminal amino acid residues, depicted as SEQ ID NO:1 (YPGSVDWRQK GAVTP).

The forty-two rounds of Edman degradation of CC23a resulted in the identification of the first 43 N-terminal amino acid residues, depicted as SEQ ID NO:2 (YPGSVDWRQK GAVTPVKDQN PCGSCWAFST VATVEGINKI VTG).

### EXAMPLE 4

### Treatment of Wound of Patient

A patient diagnosed with a non-healing wound, specifically a lesion on the skin unable to heal without intervention, is selected for treatment with the cysteine protease CC23a. The purified protease CC23a can be used at a concentration of about 200 ng/ml as a single application, twice daily for a period of about a week. The amount of topical formulation administered to a patient is an amount which applies about 25 μg/cm² of CC23a to the lesion.

After treatment for approximately a week, the non-healing lesion shows signs of undergoing healing.

What is claimed is:

1. A wound treating composition comprising an amount of a protease effective to treat a wound, wherein the protease stimulates cell proliferation and is selected from the group consisting of a natural protease isolated from genus Carica, a recombinant protease identical to the natural protease, and conservative mutations thereof.

2. The wound treating composition of claim 1, wherein the protease is a cysteine protease.

3. The wound treating composition of claim 1, wherein the protease is a natural protease isolated from Caricaceae candamarcensis.

4. The wound treating composition of claim 1, wherein the protease comprises an amino acid sequence substantially homologous with SEQ ID NO:2.

5. The wound treating composition of claim 1, wherein the amount of protease contained in a single application is from about 50 ng/ml to about 500 ng/ml.

6. The wound treating composition of claim 1, further comprising antibiotics.

7. The wound treating composition of claim 1, further comprising a protein denaturing agent.

8. A method of treating wounds comprising administering to a wound an amount of a protease effective to treat a wound, wherein the protease stimulates cell proliferation and is selected from the group consisting of a natural protease isolated from genus Carica, a recombinant protease identical to the natural protease, and conservative mutations thereof.

9. The method of treating wounds of claim 8, wherein the protease is a cysteine protease.

10. The method of treating wounds of claim 8, wherein the protease is extracted from Caricaceae candamarcensis.

11. The method of treating wounds of claim 8, wherein the protease comprises an amino acid sequence substantially homologous with SEQ ID NO:2.

12. The method of treating wounds of claim 8, wherein the amount of protease is from about 0.01 μg/cm² to about 100 μg/cm².

13. A protease isolated from genus Carica that hydrolyzes protein components of wounded tissue and stimulates cell proliferation.

14. The protease of claim 13, further comprising a cysteine protease.

15. The protease of claim 13, wherein the Carica is Carica candamarcensis.

16. The protease of claim 13, comprising an N-terminal amino acid sequence substantially homologous with SEQ ID NO:2.

17. The protease of claim 14 that has characteristics nearly identical to CC23a or CC23b.
